(19) United States
(12) Patent Application Publication
(10) Pub. No.: US 2008/0114387 A1
(43) Pub. Date: May 15, 2008

(54) Dual linear ultrasound control

(76) Inventors: David W. Hertweck, Valley Park, MO (US); Jeffery A. Knight, Maryland Heights, MO (US)

Correspondence Address: Bausch & Lomb Incorporated, One Bausch & Lomb Place, Rochester, NY, 14604-2701, US

(21) Appl. No.: 11/595,544

(22) Filed: Nov. 10, 2006

(51) Int. Cl. A61F 9/007

(52) U.S. Cl. 606/170; 606/1; 601/2

(57) ABSTRACT

An ophthalmic surgical control system 10 includes a surgical console 12 for controlling a variety of surgical instruments. A foot controller 18 is connected to the surgical console 12 including a pedal 24 for movement by a user over a pre-determined range in pitch 26 and yaw 28. The system 10 allows the foot controller 18 to independently control two parameters for a single function where a first parameter is controlled by movement of the pedal 24 in pitch 26 and a second parameter is controlled by movement of the pedal 24 in yaw 28.

### BACKGROUND OF THE INVENTION

1. Field of the Invention

The present invention is related to ophthalmic surgical control systems having foot controllers and, more particularly, to foot controllers having foot pedals with movement in both pitch and yaw.

2. Description of Related Art

The use of foot controllers in ophthalmic surgical systems is well known. The foot controllers typically have a foot pedal that pivots about an axis. As the pedal is depressed the foot controller causes a change in some operating function such as ultrasound, irrigation and aspiration, vitrectomy cutter, coagulation, or some other function. The movement or travel in pitch, i.e. up and down, of the pedal is typically divided into one or more regions. For example a total pitch movement of the pedal may be 15° with three regions contained within the 15° of movement. Continuing with the three region example, each region then forms some portion of the total 15° of movement. For example region 1 may be 5% of the pitch movement, region 2 may be 30%, and region 3 50% or more. There is typically some detent that provides tactile feedback to a user as the pedal moves from one region to the next.

In addition to pitch movement, some foot controllers provide movement in yas, i.e. side-to-side movement. These foot controllers are referred to as dual linear foot controllers. One such dual linear foot controller is described in U.S. Pat. No. 6,179,829 and assigned to the same assignee as the present invention and is hereby incorporated in its entirety by reference. The movement in yaw provides another direction of travel for a surgeon to controller further functions of surgery. The yaw movement is typically divided between left and right movements.

Such movement in pitch and yaw provides for effective control of surgical functions but some functions have multiple parameters that need to be adjusted during surgery. For example in controlling ultrasound during phacoemulsification in cataract surgery the function of ultrasound control includes such parameters as power, pulse rate, duty cycle, pulse duration, pulse interval, and minimum/maximum duty cycle switching. Prior art systems did not allow the foot controller to independently control more that one of these parameters at a time. Typically when the foot controller traveled from one region to the next the surgical system would force a parameter to a set value or a set change in value as the parameter of the current region was controlled. This was also true of dual linear foot controllers. If a surgeon wanted to change a parameter other than the one currently programmed to the foot controller, the parameter would have to be changed at the surgical console. This could lead to unwanted delay in the surgery.

Therefore, a need exists to allow independent control of two parameters of the same function to provide for more surgeon control and improve efficiency.

### BRIEF DESCRIPTION OF THE DRAWINGS

FIG. 1 is a view of a surgical control system in accordance with the present invention; and

FIG. 2 is a perspective view of a portion of FIG. 1.

### DETAILED DESCRIPTION OF THE PREFERRED EMBODIMENT

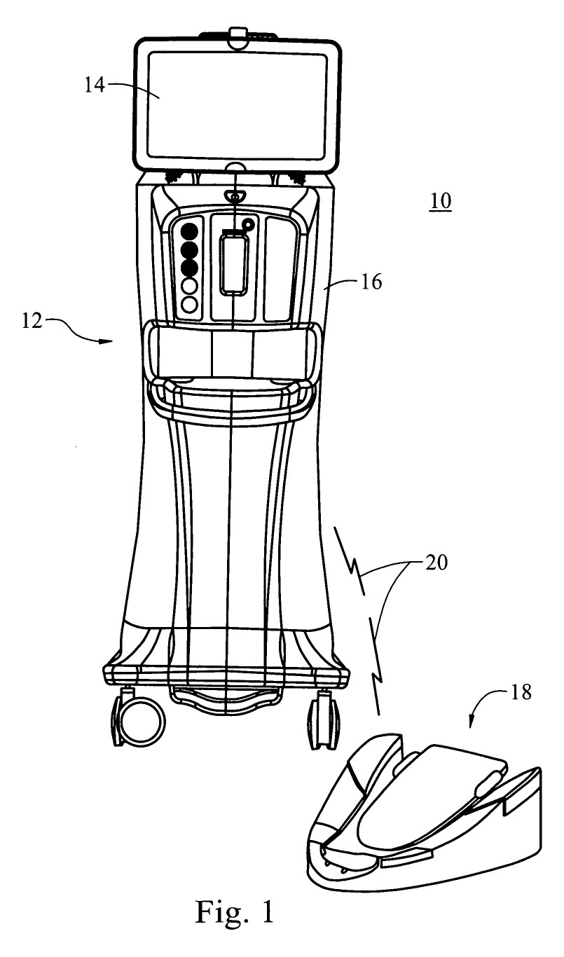

FIG. 1 shows an ophthalmic surgical control system 10 in accordance with the present invention. System 10 includes a surgical console 12 with a display 14 and control modules (not shown) within body 16. The system 10 is also connected to a foot controller 18 for movement of a pedal by a user over a pre-determined range in pitch and yaw. Foot controller 18 may be connected to the console 12 by a cable or wirelessly as indicated by lines 20. Surgical console 12 controls a variety of surgical instruments. The system 10 allows the foot controller 18 to independently control two parameters for a single function where a first parameter is controlled by movement of the pedal in pitch and a second parameter is controlled by movement of the pedal in yaw.

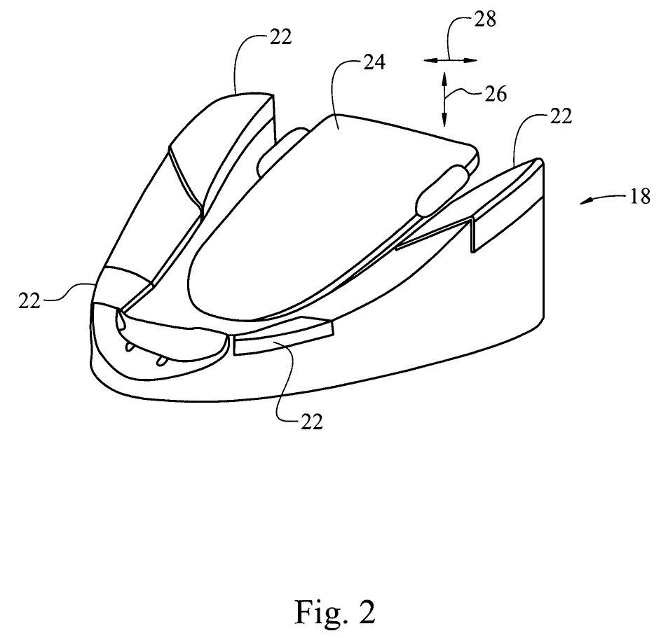

The foot controller 18, best seen in FIG. 2, typically has four buttons 22 and a center foot pedal 24, which has two axes of movement, to control two linear functions simultaneously or two parameters of a single function independently. The pedal 24 operates in both pitch (up and down travel indicated at 26) and yaw (side to side travel indicated at 28). The yaw movement simulates the side switches used on some systems, and can be set and programmed for left-foot or right-foot users. Reflux (if selected) is always activated by inward yaw displacement of pedal 24. The pedal 24 may be programmed to operate two linear functions simultaneously (Dual Linear control). The control of linear functions is proportional to the amount of pedal travel. In single linear mode, pitch controls the linear functions selected, and yaw provides mode, pitch controls the liner functions selected, and yaw movements provides on/off control in both directions.

The pedal 24, located in the center of the foot control, provides two axes of movement and thus allows simultaneous independent control of two system parameters. Both pedal movements are programmable with respect to function and control parameters. In the pitch direction, pedal 24 provides approximately 15° of up/down movement. In the yaw direction, pedal provides approximately 10° of travel from center in both the left and right directions, however, the center (home) position may be offset approximately 5° in either direction. When released, pedal 24 returns to the home (up or center) position. The foot controller 18 includes programmable detents (not shown) that provide tactile feedback to the pitch movement when it moves between different regions.

The following are examples of foot controller 18 configurations>Single Region Pitch Control (one detent position)

The pitch movement is programmed to provide linear control as a function of relative pedal 24 displacement (e.g. 0° to 15° down corresponds to 0% to 100% output). An example of single region pitch control is the linear coagulation function.

### Two Region Pitch Control

There are two programmable regions (two detent positions). When programmed for linear control, the pitch movement is a function of relative pedal 2 displacement in region 2 (e.g., 5° to 15° down corresponds to 0% to 100% output). An example is irrigation/aspiration (I/A) control, where region 1 is for irrigation and region 2 is for linear vacuum or flow.

### Three Region Pitch Control

There are three programmable regions (three detent positions). When programmed for linear control, pitch movement is a function of relative pedal 24 displacement as shown below. An example is single linear ultrasound phases, where region 1 is irrigation, region 2 is linear aspiration, and region 3 is linear power.

### Programmable Yaw Positions

The foot controller 18 may be set and programmed to give greater linear yaw movement for either right or left foot operation.

### Single Linear Setup

In vitrectomy mode, the outward yaw movement provides ON/OFF cutting control. Each successive outward movement toggles the programmed tool ON or OFF. In ultrasound mode, outward yaw control could be programmed to toggle between different ultrasound submodes. When pedal 24 is released, it returns to the center position. Inward yaw movement controls reflux.

### Dual Linear Setup

The outward yaw movement provides linear control of the programmed function, relative to pedal 24 displacement (e.g., 0° to 15° displacement corresponds to 0% to 100% output). When pedal 24 is released, it returns to the center position. Inward yaw movements controls reflux.

### Yaw Control of Reflux

The pedal 24 may be programmed for use with either the right or left foot. Reflux (if selected) is always activated by inward yaw displacement. For a right foot configuration, reflux is the left (inward). For a left foot configuration, reflux would be to the right. Reflux may only be activated when aspiration is not activated.

### Yaw Control of Ultrasound Submode

For single linear setup, the ultrasound submode sequence (if programmed) is activated by inward or outward yaw when the pedal 24 is in region 2 or region 3. In a Dual Linear setup, the yaw control of the ultrasound submode can only be activated (if programmed) by inward yaw when pedal 24 is in region 2 or region 3.

The present invention allows for the use the two axis of dual linear foot controller 18 to vary two parameters associated with, for example, the ultrasound function for cataract removal. Ultrasound modulations can be described by various parameters such as power, pulse rate, duty cycle, pulse duration, and pulse interval. Typical examples are:
Pulsed Mode: Power, Pulse Rate, and Duty Cycle
Pulsed Mode: Power, Pulse Duration, and Pulse Interval
Multiple Burst Mode: Power, Pulse Duration, and Minimum/Maximum Duty Cycle

The present invention allows two of the three parameters in the above exemplary modes to be controlled with the pitch and yaw axis of pedal 24. The modes provided are:

### Linear Power Linear Pulse:

Power is varied between and minimum and a maximum with foot controller 18 motion in either pitch or yaw,
Pulse Rate is varied between and minimum and a maximum with foot controller 18 motion in the other of pitch or yaw not selected for power control,
Duty Cycle is a fixed console setting.

In each of the following examples as in the above example the first parameter to be varied may be set for either pitch or yaw and the second parameter is then set to be varied in the direction other than that chosen for the first parameter.

### Linear Power Linear Duty Cycle

Power is varied between a minimum and a maximum with foot controller 18 motion,
Pulse Rate is a fixed console setting,
Duty Cycle is varied between a minimum and a maximum with foot controller 18 motion.

### Dual Linear Multiple Burst

Power is a fixed console setting,
Burst Duration is varied between a minimum and a maximum with foot controller 18 motion,
Duty Cycle is varied between a minimum and a maximum with foot controller 18 motion.

### Variable Power Multiple Burst

Power is varied between a minimum and a maximum with foot controller 18 motion,
Burst Duration is a fixed console setting,
Duty Cycle is varied between a minimum and a maximum with foot controller 18 motion.

### Variable Power Linear Burst

Power is varied between a minimum and a maximum with foot controller 18 motion,
Burst Duration is varied between a minimum and a maximum with foot controller 18 motion,
Burst Interval is a fixed console setting.

The mapping from the foot controller 18 position to the parameter set point may be linear or non-linear and also may be reversed, such that the minimum foot pedal travel produces the maximum parameter value.

Thus, there has been shown a control system that allows independent control of two parameters of a single function with the pedal of a foot controller. Prior art system only allowed a single parameter of a function to be controlled with the pedal. All parameters but one were fixed console settings in prior art systems. By allowing two parameters to be independently controlled by the foot controller the surgeon has greater flexibility and control, which should result in more efficient surgery.

In addition to the above example of control of the function of ultrasound it is possible to independently control two parameters of other function such as vitrectomy cutters. Control of vitrectomy cutter parameters includes cut rate, duty cycle, min/max cut rate, and burst duration and rate. Another function that may be controlled is fluid flow, where the parameters include at least irrigation and aspiration. Other functions and their associated parameters may be apparent to those skilled in the art and are intended to be within the scope of the present invention.

Additional control modes are possible including modes where multiple parameters may be controlled on the same axis of motion, each with their own mapping from foot control position to parameter value. In addition, it is also possible to have modes where any of the parameters may be set to vary between a minimum and a maximum value automatically over time.

What is claimed:

1. An ophthalmic surgical control system comprising:
a surgical console for controlling a variety of surgical instruments;
a foot controller connected to the surgical console including a pedal for movement by a user over a pre-determined range in pitch and yaw;
wherein the system allows the foot controller to independently control two parameters for a single function where a first parameter is controlled by movement of the pedal in pitch and a second parameter is controlled by movement of the pedal in yaw.

2. The system of claim 1, wherein the foot controller is connected to the console wirelessly.

3. The system of claim 1, wherein the pedal moves in pitch approximately fifteen degrees and the pedal moves in yaw approximately 10 degrees.

4. The system of claim 1, wherein the function to be controlled includes ultrasound and the parameters include at least power, pulse rate, duty cycle, pulse duration, and pulse interval.

5. The system of claim 1, wherein the function to be controlled includes fluid flow and the parameters includes at least irrigation and aspiration.

6. The system of claim 1, wherein the function to be controlled includes vitrectomy cutting and the parameters include at least cut rate, duty cycle, min/max cut rate, and burst duration and rate.

7. A method of operating an ophthalmic surgical control system comprising the steps of:
providing a surgical console and controlling a variety of surgical instruments;
connecting a foot controller to the surgical console, the foot controller including pedal for movement by a user over a pre-determined range in pitch and yaw; and
independently controlling with the foot controller two parameters of a single function where a first parameter is controlled by movement of the pedal in pitch and a second parameter is controlled by movement of the pedal in yaw.

8. The method of claim 7, wherein the connecting step includes connecting the foot controller to the console wirelessly.

9. The system of claim 7, wherein the function to be controlled includes ultrasound and the parameters include at least power, pulse rate, duty cycle, pulse duration, and pulse interval.

10. The system of claim 7, wherein the function to be controlled includes fluid flow and the parameters includes at least irrigation and aspiration.

11. The system of claim 7, wherein the function to be controlled includes vitrectomy cutting and the parameters include at least cut rate, duty cycle, min/max cut rate, and burst duration and rate.
